(19) United States
(12) Patent Application Publication
(10) Pub. No.: US 2020/0337528 A1
(43) Pub. Date: Oct. 29, 2020

(54) INSERTION APPARATUS

(71) Applicant: OLYMPUS CORPORATION, Tokyo (JP)

(72) Inventor: Yoshitaka NAKANO, Tokyo (JP)

(73) Assignee: OLYMPUS CORPORATION, Tokyo (JP)

(21) Appl. No.: 16/928,134

(22) Filed: Jul. 14, 2020

Related U.S. Application Data
(63) Continuation of application No. PCT/JP2018/033271, filed on Sep. 7, 2018.

(30) Foreign Application Priority Data
Jan. 29, 2018 (JP) 2018-012706

(51) Int. Cl. A61B 1/00; A61B 1/018; A61B 1/005; A61B 1/307

(52) U.S. Cl. CPC A61B 1/00078; A61B 1/307; A61B 1/0055; A61B 1/018

(57) ABSTRACT

An insertion apparatus includes an insertion section, a channel tube, a tube bending part, and a protective member disposed at the tube bending part, the protective member being configured to prevent deformation of the tube bending part when a guide wire passes through the tube bending part.

### CROSS REFERENCE TO RELATED APPLICATION

This application is a continuation application of PCT/JP2018/033271 filed on Sep. 7, 2018 and claims benefit of Japanese Application No. 2018-012706 filed in Japan on Jan. 29, 2018, the entire contents of which are incorporated herein by this reference.

### BACKGROUND OF THE INVENTION

### 1. Field of the Invention

The present invention relates to an insertion apparatus provided with a tube bending part having a shape which bends an advancing direction of an elongated member which is inserted into a channel tube from a distal end opening. The tube bending part is formed at an intermediate portion of the channel tube which passes through an insertion section.

### 2. Description of the Related Art

Recently, an insertion apparatus, for example, an endoscope has been popularly used in a medical field. The endoscope enables observation, treatment and the like of a site to be examined in an organ in a body which is a subject by inserting an elongated insertion section into the organ.

In this case, for example, when the organ is a kidney, a technique has been well known where an insertion section of an endoscope for a renal pelvis (hereinafter, referred to as renal pelvic ureter endoscope) having an extremely small diameter of approximately 3 mm is inserted into a ureter, and the insertion section is inserted into the kidney through the ureter and, thereafter, a ureteral stone in the kidney is observed or removed.

In such an operation, to enhance insertability of the insertion section into the kidney through the ureter, a technique is also well known where, first, a known guide wire which is an elongated member having a smaller diameter than the insertion section of the renal pelvic ureter endoscope is inserted into the kidney through the ureter and, thereafter, the guide wire is inserted into a flexible channel tube inserted into the insertion section of the renal pelvic ureter endoscope from a distal end opening of the channel tube, that is, a technique where the insertion section is inserted into the kidney along the guide wire inserted into the channel tube.

In such a renal pelvic ureter endoscope, the above-mentioned insertion section is formed with an extremely small diameter of 3 mm, for example. Accordingly, when the channel tube inserted into the insertion section is inserted straightly along a longitudinal axis of the insertion section from the distal end opening positioned in an offset manner in a radial direction from the center of the insertion section, the channel tube is caught by an inner peripheral surface of a bending piece which configures a bending portion formed on the insertion section and hence, a bending performance is lowered.

In view of the above, to ensure a bending performance of the bending portion while realizing downsizing of the insertion section, a configuration is well known where a portion of the channel tube which is disposed on a more proximal end side than a rigid internal component, for example, an image pickup unit disposed in a distal end side of the insertion section is disposed close to the center of the insertion section.

In other words, the channel tube having the following configuration is well known. A tube bending part is formed at an intermediate portion of the channel tube, specifically at a portion of the channel tube in the vicinity of a proximal end of an image pickup unit. The tube bending part is bent in a crank shape from the outside to the center of the insertion section in a radial direction so that an advancing direction of the guide wire inserted into the channel tube is bent.

Japanese Patent Application Laid-Open Publication No. 6-54799 discloses the configuration of a distal end side of an endoscope where a tube bending part is formed of a stainless steel pipe and hence, breaking of the tube bending part caused by insertion of an elongated member is prevented.

In the configuration disclosed in Japanese Patent Application Laid-Open Publication No. 6-54799, the stainless steel pipe is rigid and hence, breaking of the tube bending part can be prevented with certainty.

### SUMMARY OF THE INVENTION

According to an aspect of the present invention, there is provided an insertion apparatus which includes: an insertion section inserted into a subject; a channel tube formed using a deforming material, the channel tube being disposed at a distal end of the insertion section and substantially parallel to a longitudinal axis of the insertion section, the channel tube passing through the insertion section, the channel tube having a distal end opening through which an elongated member is inserted at the distal end of the insertion section; a tube bending part formed at an intermediate portion of the channel tube, the tube bending part having a shape which bends an advancing direction of the elongated member inserted into the channel tube from the distal end opening; and a protective member disposed at the tube bending part, the protective member having a portion which is positioned spaced apart from a center of the insertion section toward an outside in a radial direction of the insertion section, with respect to an outer peripheral surface of a portion of the channel tube on a more distal end side than the tube bending part, the protective member being configured to prevent deformation of the tube bending part when the elongated member passes through the tube bending part.

### BRIEF DESCRIPTION OF THE DRAWINGS

FIG. 1 is a partial perspective view showing an endoscope according to the present embodiment;

FIG. 2 is a cross-sectional view showing the schematic configuration of a distal end side of an insertion section taken along a line 11-1 in FIG. 1;

FIG. 3 is a view schematically showing the configuration of the distal end side of the insertion section shown in FIG. 2 where a protective member is mainly described;

FIG. 4 is a view showing the configuration of the distal end side of the insertion section shown in FIG. 3 together with a modification of an inclined surface of the protective member;

FIG. 5 is a view showing a modification where the protective member shown in FIG. 3 is positioned spaced apart rearward from an outer peripheral surface of a tube bending part in a longitudinal axis direction; and

FIG. 6 is a view schematically showing the configuration of a distal end side of a conventional insertion section where a tube bending part and a protective member are mainly described.

### DETAILED DESCRIPTION OF THE PREFERRED EMBODIMENT

Hereinafter, an embodiment of the present invention is described with reference to drawings. Drawings are schematic views. Accordingly, it must be noted that a relationship between a thickness and a width of each portion, a ratio between thicknesses of respective portions and the like differ from the corresponding relationships of portions of an actual insertion apparatus. Needless to say, there are portions described with different size relationship or different ratios between the drawings.

In an embodiment described hereinafter, an insertion apparatus is described by taking an example where an endoscope forms an insertion apparatus.

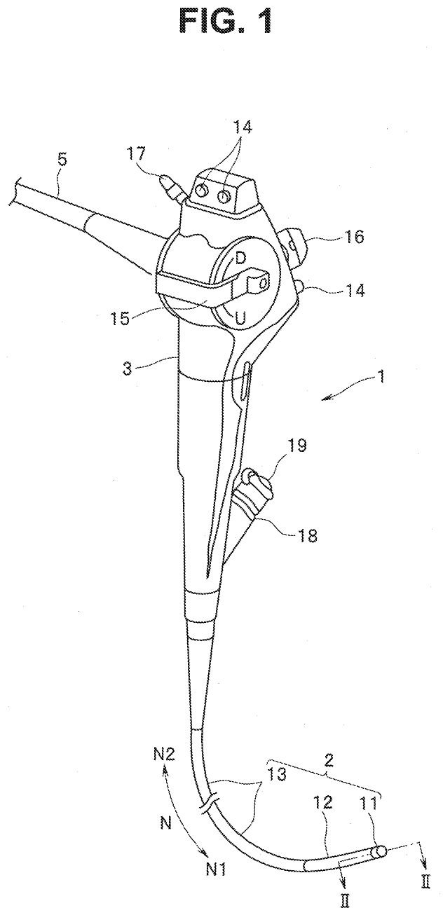

FIG. 1 is a partial perspective view showing an endoscope according to the present embodiment.

As shown in FIG. 1, the endoscope 1 includes: as main sections, an insertion section 2 which is elongated in a direction along a longitudinal axis (hereinafter, also referred to as a longitudinal axis direction) N, has flexibility and is inserted into a subject, an operation section 3 disposed on a proximal end side of the insertion section 2 in the longitudinal axis direction N (hereinafter, simply referred to as a proximal end side); a universal cord 5 which extends from the operation section 3; and a connector (not shown) which is connected to an image processing apparatus, a light source apparatus and the like (not shown) which are disposed on an extension end of the universal cord.

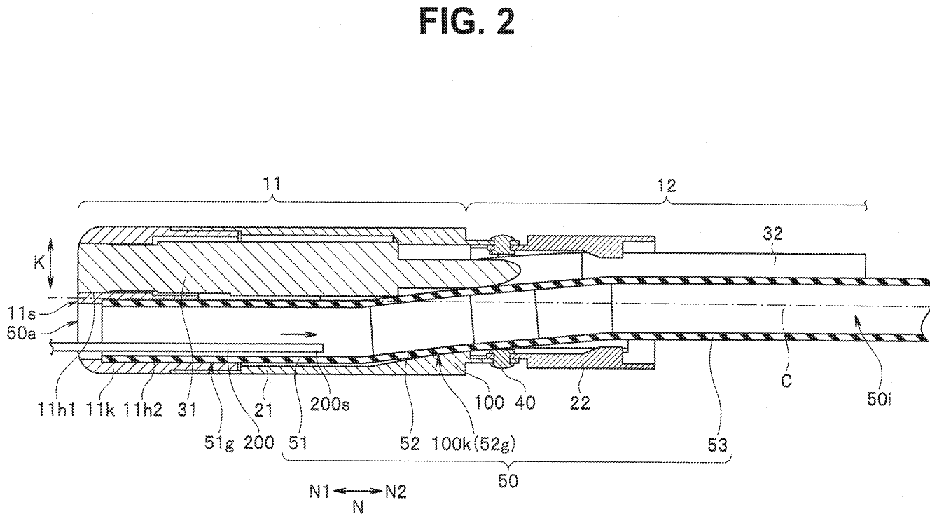

The insertion section 2 mainly includes, in the order from a distal end side in the longitudinal axis direction N (hereinafter, simply referred to as a distal end side): a rigid distal end portion 11 in which an image pickup unit 31 (see FIG. 2) and the like described later are disposed; a bending portion 12 which is disposed on a proximal end side of the distal end portion 11, and is capable of bending a distal end side of the insertion section 2 in a plurality of directions, for example, two directions (upward and downward directions); and a soft flexible tube portion 13 which is disposed on a proximal end side of the bending portion 12 and has flexibility. The insertion section 2 is formed in a small diameter. As the endoscope 1 having such a small-diameter insertion section 2, a renal pelvic ureter endoscope is named, for example.

On a proximal end side of the operation section 3, remote switches 14 for performing image control instructions such as freeze and release, a bending operation lever 15 for performing a bending operation of the bending portion 12, a suction button 16 for performing a suction operation, a suction pipe sleeve 17 which communicates with a channel tube 50 (see FIG. 2) passing through the insertion section 2 and the operation section 3 described later and the like are arranged.

On a distal end side of the operation section 3, a treatment instrument insertion opening 18 which allows the insertion of a treatment instrument into the channel tube 50 is disposed, and a forceps plug 19 is detachably attached in the treatment instrument insertion opening 18.

Next, the structure of the distal end side of the insertion section 2 is described with reference to FIG. 2. FIG. 2 is a cross-sectional view showing the schematic configuration of the distal end side of the insertion section taken along a line I-II in FIG. 1.

As shown in FIG. 2, the distal end portion 11 includes a distal end rigid member 11k on the distal end side of the insertion section 2.

Among a plurality of bending pieces which configure the bending portion 12, a distal end side of the bending piece 21 positioned on a most distal end side is fixed by fitting to an outer periphery of the distal end rigid member 11k on a proximal end side.

A distal end side of the bending piece 22 which configures the bending portion 12 is fitted on an inner periphery of the bending piece 21 on a proximal end side, and the bending piece 22 is connected to the bending piece 21 by rivets 40 such that the bending portion 12 is rotatable in two directions, for example, upward and downward directions.

In an actual structure, a plurality of bending pieces are successively connected to each other in series in the longitudinal axis direction N by way of rivets from the proximal end side of the bending piece 22. However, for the sake of brevity of explanation of drawings, the description of these bending pieces is omitted.

A distal end side of the image pickup unit 31 which includes an observation optical system and an image pickup device and is a rigid internal component is fitted in and fixed to an arrangement hole 11h1 formed in the distal end rigid member 11k in the longitudinal axis direction N such that the image pickup unit 31 picks up an image of a subject positioned on a more front side N1 than a distal end surface 11s in the longitudinal axis direction N. In other words, the endoscope 1 according to the present embodiment is a known front-view type endoscope.

An image pickup cable 32 having a smaller diameter than the image pickup unit 31 extends from the image pickup unit 31 toward a rear side N2 in the longitudinal axis direction N. The image pickup cable 32 extends to the above-mentioned connector (not shown) of the endoscope 1 by way of the insertion section 2, the operation section 3, and the universal cord 5.

A distal end side of a channel tube 50 which is formed using a flexible deforming material and passes through the insertion section 2 and the operation section 3 substantially parallel to the longitudinal axis direction N is fitted in and fixed to an arrangement hole 11h2 formed in the distal end rigid member 11k in the longitudinal axis direction N.

The channel tube 50 is divided in the operation section 3, and one divided portion on a proximal end side opens in a suction pipe sleeve 17, and the other divided portion on a proximal end side opens in a treatment instrument insertion opening 18.

A distal end side of the channel tube 50 opens on the distal end surface 11s of the distal end portion 11. In other words, the channel tube 50 has a distal end opening 50a at the distal end surface 11s.

A channel 50i is formed in the channel tube 50. The channel 50i forms a space through which a liquid or solid body passes through from the distal end opening 50a toward the rear side N2 at the time of sucking a liquid, a solid body or the like of a subject from the distal end opening 50a by a suction apparatus (not shown) connected to the suction pipe sleeve 17. Further, a treatment instrument (not shown) passes through the space toward a front side N1 in the longitudinal axis direction N at the time of inserting the treatment instrument into a subject from the treatment instrument insertion opening 18 through the distal end opening 50a. Still further, a guide wire 200 which is an elongated member is inserted into the space toward the rear side N2 from the distal end opening 50a.

A distal end side of the channel tube 50 is formed of a tube bending part 52; a part disposed on a more distal end side than the tube bending part 52 (hereinafter, referred to as a distal end side part) 51; and a part disposed on a more proximal end side than the tube bending part 52 (hereinafter, referred to as a proximal end side part) 53.

The distal end side part 51 has the distal end opening 50a, extends in the longitudinal axis direction N, and is disposed in the vicinity of the image pickup unit 31 in a radial direction K of the insertion section 2. Note that the distal end opening 50a is positioned in an offset manner in the radial direction K with respect to a center C of the insertion section 2.

The tube bending part 52 is disposed at an intermediate portion of a distal end side of the channel tube 50 in the longitudinal axis direction N.

More specifically, the tube bending part 52 is disposed on a more rear side N2 than the distal end rigid member 11k, and has a shape which bends an advancing direction of the guide wire 200 inserted into the channel 50i from the distal end opening 50a. More specifically, the tube bending part 52 is formed in a meandering shape where the tube bending part 52 is bent in a crank shape.

The proximal end side part 53 extends in the longitudinal axis direction N, and is disposed close to the center C of the insertion section and in the vicinity of the image pickup cable 32 in the radial direction K. In other words, the proximal end side part 53 is disposed closer to the center C than the distal end side part 51 in the radial direction K by the tube bending part 52.

More specifically, the proximal end side part 53 is disposed closer to the center C than the distal end side part 51 in the radial direction K by the tube bending part 52 by a difference between a diameter of the image pickup unit 31 and a diameter of the image pickup cable 32.

With such a configuration, downsizing of the insertion section 2 can be realized and, at the same time, it is possible to prevent the proximal end side part 53 from being brought into contact with inner peripheral surfaces of the above-mentioned plurality of bending pieces in the bending portion 12.

In the distal end portion 11, a protective member 100 which prevents the deformation of the guide wire 200 when the guide wire 200 passes in the tube bending part 52 is disposed on the tube bending part 52 at a position on a more rear side N2 than the image pickup unit 31.

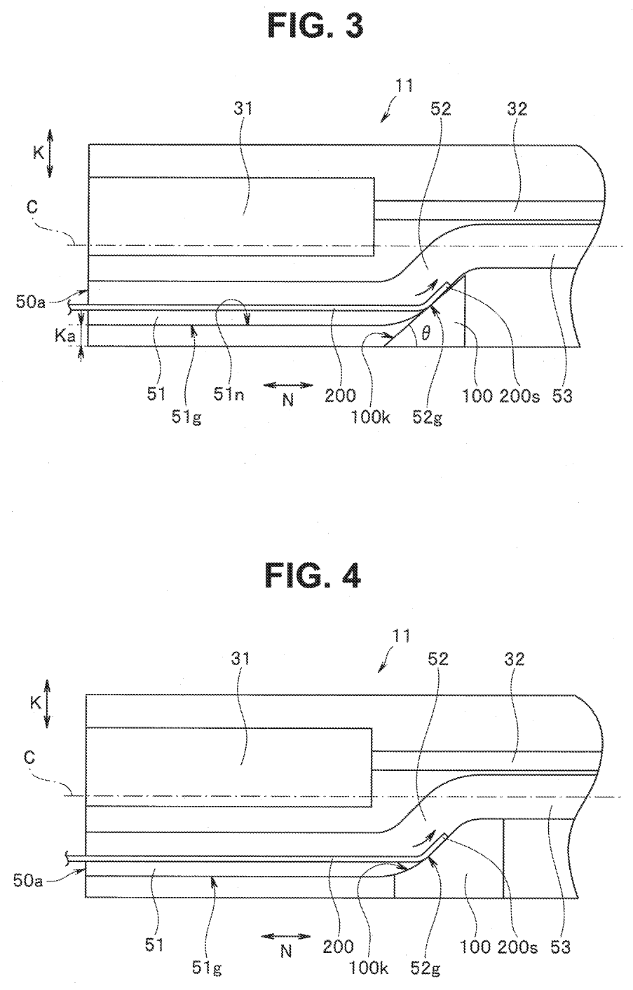

The detailed configuration of the protective member 100 is described with reference to FIG. 3 to FIG. 6.

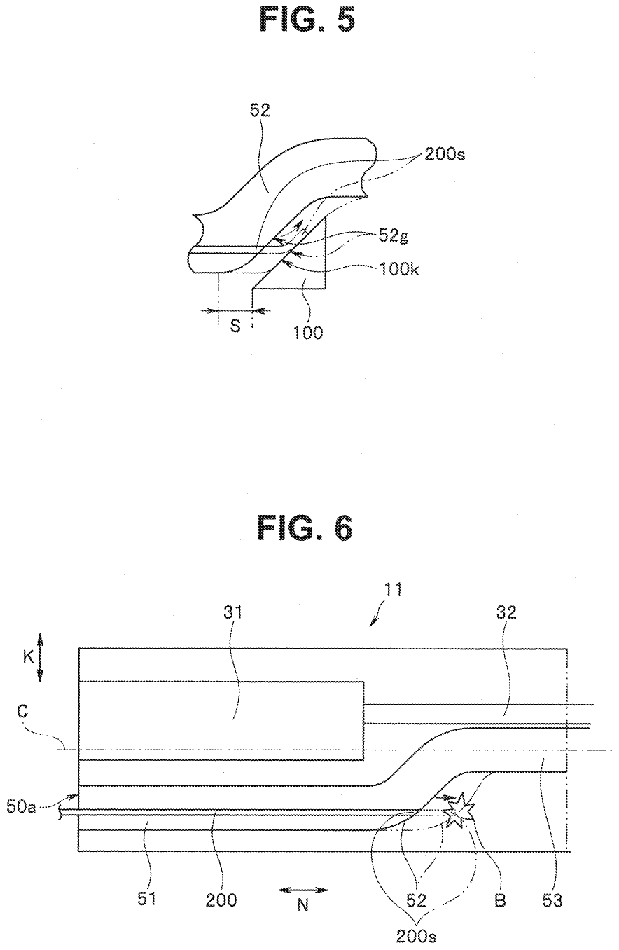

FIG. 3 is a view schematically showing the configuration of the distal end side of the insertion section shown in FIG. 2 where the protective member is mainly described. FIG. 4 is a view showing the configuration of the distal end side of the insertion section shown in FIG. 3 together with a modification of an inclined surface of the protective member. FIG. 5 is a view showing a modification where the protective member shown in FIG. 3 is positioned spaced apart rearward from an outer peripheral surface of a tube bending part in a longitudinal axis direction. FIG. 6 is a view schematically showing the configuration of a distal end side of a conventional insertion section where a tube bending part and a protective member are mainly described.

As shown in FIG. 3, the protective member 100 is disposed in the insertion section 2 on the distal end side. The protective member 100 is disposed on a more proximal end side than the image pickup unit 31, and is also disposed on an outer peripheral surface 52g of the tube bending part 52.

More specifically, the protective member 100 is positioned on a bent shape part of the outer peripheral surface 52g of the tube bending part 52 positioned on a side spaced apart from the center C in the radial direction K.

As shown in FIG. 2 and FIG. 3, the protective member 100 is formed in a shape having an approximately triangular cross section. The protective member 100 has an inclined surface 100k which is inclined with respect to the longitudinal axis direction N so as to face the distal end opening 50a.

More specifically, the inclined surface 100k is formed in a planar shape inclined linearly such that a distance between the inclined surface 100k and the center C of the insertion section 2 in the radial direction K is decreased as the inclined surface 100k extends toward the rear side N2.

More specifically, an inclination angle θ of the inclined surface 100k with respect to the longitudinal axis direction N may be set in conformity with a bending amount of the tube bending part 52 in the radial direction K, that is, in conformity with a difference in the radial direction K between the center of an opening of the tube bending part 52 on a distal end side part 51 side and the center of an opening of the tube bending part 52 on a proximal end side part 53 side, or may be set in conformity with a bent shape of the tube bending part 52.

However, as described later, the inclined surface 100k has a function of guiding a distal end 200s of the guide wire 200 which is brought into contact with the tube bending part 52 toward the proximal end side part 53. Accordingly, when the inclination angle θ is set to an angle close to 90°, the guide wire 200 cannot be guided. As a result, it is necessary to set the inclination angle θ to a value which allows the deformation of the distal end 200s of the guide wire 200.

By taking into account the above-mentioned setting of the inclination angle θ, the cross-sectional shape of the protective member 100 is not limited to a triangular cross-sectional shape, and other shapes may be used. In other words, the inclined surface 100k is not limited to a linear shape, and may be a spherical shape or a bent shape as shown in FIG. 4.

The protective member 100 is provided for preventing the occurrence of a following case The distal end 200s of the guide wire 200 inserted in the distal end side part 51 from the distal end opening 50a impinges on the tube bending part 52 and deforms the tube bending part 52. For example, the distal end 200s of the guide wire 200 impinges on and raises the tube bending part 52 toward the rear side N2 and, as shown in FIG. 6, the tube bending part 52 of the channel tube 50 formed using a flexible deforming material is broken at an impinging position B.

In other words, the protective member 100 suppresses the deformation of the tube bending part 52 caused when the distal end 200s of the guide wire 200 is brought into contact with the tube bending part 52.

Further, the protective member 100 is also a member which guides a distal end 200s side of the guide wire 200 which impinges on the tube bending part 52 toward a proximal end side of the tube bending part 52, that is, toward a proximal end side part 53 side by the inclined surface 100k. In other words, the protective member 100 is a member which smoothly deforms the distal end 200s side of the guide wire 200 into a bent shape of the tube bending part 52 by the inclined surface 100k.

Accordingly, as shown in FIG. 3, the protective member 100 may be disposed in contact with the outer peripheral surface 52g. Further, as shown in FIG. 5, the protective member 100 may be disposed such that the protective member 100 is disposed in a spaced apart manner from the outer peripheral surface 52g toward the rear side N2 by a distance S, and the outer peripheral surface 52g of the tube bending part 52 which is raised by impingement of the guide wire 200 is brought into contact with the protective member 100 as indicated by a chain line.

When the distance S between the protective member 100 and the outer peripheral surface 52g is excessively large, the tube bending part 52 is broken before the outer peripheral surface 52g is brought into contact with the inclined surface 100k and hence, it is necessary to set the distance S to a distance that such breaking of the tube bending part 52 is prevented.

Further, as shown in FIG. 3, it is preferable that the inclined surface 100k of the protective member 100 have a portion which is positioned more spaced apart from the center C of the insertion section 2 toward the outside than an outer peripheral surface 51g of the distal end side part 51 in the radial direction K.

It is because that, in the case where the inclined surface 100k is positioned closer to the center C than the outer peripheral surface 51g in the radial direction K when the guide wire 200 is inserted toward the rear side N2 along the inner peripheral surface 51n of the distal end side part 51, the outer peripheral surface 52g which is raised by the distal end 200s of the guide wire 200 by impingement cannot be brought into contact with the inclined surface 100k.

In other words, such configuration is preferable to bring the distal end 200s of the guide wire 200 which raises the tube bending part 52 by impingement into contact with the inclined surface 100k with certainty.

As shown in FIG. 2, it is preferable that the protective member 100 be formed integrally with the bending piece 21 so as to realize downsizing of the diameter of the insertion section 2.

However, the protective member 100 may be formed as a separate member from the bending piece 21, and may be fixed to an inner peripheral surface of the bending piece 21.

Other configurations are equal to the corresponding configurations of the conventional endoscope and hence, the description of other configurations is omitted.

In this manner, according to the present embodiment, the description has been made with respect to the case where the protective member 100 which is brought into contact with the tube bending part 52 is disposed in the distal end side of the insertion section 2 of the endoscope 1.

According to the above-mentioned configuration, even in the case where, for realizing downsizing of the diameter of the insertion section 2, the tube bending part 52 which bends the advancing direction of the channel tube 50 toward the center C side in the radial direction K is disposed at the intermediate position of the channel tube 50 which is flexible and deformable from the distal end side part 51 toward the proximal end side part 53, the distal end 200s of the guide wire 200 inserted from the distal end opening 50a is brought into contact with the tube bending part 52, the distal end 200s of the guide wire 200 is deformed by the inclined surface 100k of the protective member 100.

Accordingly, the guide wire 200 is guided so as to advance toward the proximal end side part 53 by the inclined surface 100k. Accordingly, it is possible to allow the guide wire 200 to smoothly pass through the tube bending part 52 without causing breaking of the portion of the tube bending part 52 on which the distal end 200s of the guide wire 200 impinges.

The protective member 100 is formed on the bending piece 21 and hence, even when the protective member 100 is provided, there is no possibility that a diameter of the insertion section 2 at the portion where the protective member 100 is provided is increased.

Further, with the use of the protective member 100, compared to the case where the tube bending part 52 is formed using a stainless steel pipe as in the case of the conventional endoscope, a length of a rigid portion of the bending piece 21 in the longitudinal axis direction N can be shortened and hence, bendability of the bending portion 12 can be enhanced.

As has been described above, it is possible to provide the endoscope 1 which includes the configuration where breaking of the tube bending part 52 can be prevented without increasing the diameter of the insertion section 2.

In the embodiment described above, the guide wire 200 is described as an example of an elongated member. However, the elongated member is not limited to the guide wire 200. Needless to say, the elongated member may be an elongated member such as a cleaning brush provided that the elongated member is used in an insertion apparatus where the elongated member advances in the channel tube 50 toward the rear side N2 of the channel 50i through the distal end opening 50a.

In the present embodiment, the image pickup unit 31 is described as an example of a rigid internal component disposed in the distal end side of the insertion section 2. However, the rigid internal component is not limited to the image pickup unit 31. Needless to say, the internal component may be other rigid internal component such as an illumination unit.

In the present embodiment, the endoscope 1 is described as an example of the insertion apparatus. However, the insertion apparatus is not limited to the endoscope 1. The present invention is also applicable to an elongated member.

For example, the present invention is also applicable to an insertion apparatus which is formed using only the channel tube 50 having the illumination unit and the tube bending part 52 but not having the image pickup unit 31, and is inserted into a subject separately from the endoscope.

The present embodiment is particularly useful as an insertion apparatus having an insertion section with an extremely small diameter such as the insertion apparatus where the channel tube 50 has the tube bending part 52

What is claimed is:

1. An insertion apparatus comprising:
an insertion section inserted into a subject;
a channel tube formed using a deforming material, the channel tube being disposed at a distal end of the insertion section and substantially parallel to a longitudinal axis of the insertion section, the channel tube passing through the insertion section, the channel tube having a distal end opening through which an elongated member is inserted at the distal end of the insertion section;
a tube bending part formed at an intermediate portion of the channel tube, the tube bending part having a shape which bends an advancing direction of the elongated member inserted into the channel tube from the distal end opening; and
a protective member disposed at the tube bending part, the protective member having a portion which is positioned spaced apart from a center of the insertion section toward an outside in a radial direction of the insertion section, with respect to an outer peripheral surface of a portion of the channel tube on a more distal end side than the tube bending part, the protective member being configured to prevent deformation of the tube bending part when the elongated member passes through the tube bending part.

2. The insertion apparatus according to claim 1, wherein the protective member is disposed on an outer peripheral surface of the tube bending part in the insertion section, and has an inclined surface which is inclined with respect to the longitudinal axis such that the inclined surface faces the distal end opening.

3. The insertion apparatus according to claim 2, wherein the protective member is disposed so as to be substantially brought into contact with the outer peripheral surface of the tube bending part.

4. The insertion apparatus according to claim 1, wherein a portion of the channel tube on a more proximal end side than the tube bending part is disposed close to the center of the insertion section in the radial direction of the insertion section.

5. The insertion apparatus according to claim 4, wherein the tube bending part is disposed in a bending manner in a crank shape.

6. The insertion apparatus according to claim 4, wherein the protective member is disposed more on a proximal end side than a rigid internal component disposed in a distal end of the insertion section.

7. The insertion apparatus according to claim 1, wherein a bending piece which bends the distal end side of the insertion section in a plurality of directions is disposed in a distal end side of the insertion section, and
the protective member is disposed in the bending piece.

8. The insertion apparatus according to claim 1, wherein the insertion apparatus is an endoscope.
